(19) United States
(12) Patent Application Publication
(10) Pub. No.: US 2026/0183395 A1
(43) Pub. Date: Jul. 2, 2026

(54) COMBINED THERAPIES USING IMMUNOMODULATING DRUGS

(71) Applicant: ONK THERAPEUTICS LIMITED, Galway (IE)

(72) Inventors: Michael O'DWYER, Galway (IE); Bruce MCCREEDY, Raleigh, NC (US); Subhashis SARKAR, San Diego, CA (US); Hui ZHI, San Diego, CA (US)

(73) Assignee: ONK THERAPEUTICS LIMITED, Galway (IE)

(21) Appl. No.: 19/128,760

(22) PCT Filed: Nov. 9, 2023
(86) PCT No.: PCT/EP2023/081326

§ 371 Date: May 9, 2025

(30) Foreign Application Priority Data
Nov. 10, 2022 (EP) 22206622.7

(51) Int. Cl. A61K 40/31; A61K 31/454; A61K 31/5377; A61K 40/15; A61K 40/35; A61K 40/42; A61P 35/00

(52) U.S. Cl. CPC A61K 40/31; A61K 31/454; A61K 31/5377; A61K 40/15; A61K 40/35; A61K 40/4205; A61K 40/4211; A61K 40/4222; A61K 40/4224; A61K 40/4257; A61P 35/00

(57) ABSTRACT

Methods of cancer treatment comprise administration of a modified natural killer (NK) cell in combination with an immunomodulatory drug. Also described are compositions comprising a combination of a modified NK cell and an immunomodulatory drug.

### INTRODUCTION

The present invention relates to the modification of natural killer (NK) cells and NK cell lines to produce derivatives thereof with a more cytotoxic phenotype. Furthermore, the present invention relates to methods of producing modified NK cells and NK cell lines, compositions containing the cells and cell lines and uses of said cells, lines and compositions in the treatment of cancer.

### BACKGROUND TO THE INVENTION

Typically, immune cells require a target cell to present antigen via major histocompatibility complex (MHC) before triggering an immune response resulting in the death of the target cell. This allows cancer cells not presenting MHC class I to evade the majority of immune responses.

NK cells, however, are able to recognize cancer cells in the absence of MHC class I expression. Hence they perform a critical role in the body's defence against cancer.

On the other hand, in certain circumstances, cancer cells demonstrate an ability to dampen the cytotoxic activity of NK cells, through expression of ligands that bind inhibitory receptors on the NK cell membrane. Resistance to cancer can involve a balance between these and other factors.

Cytotoxicity, in this context, refers to the ability of immune effector cells, e.g. NK cells, to induce cancer cell death, e.g. by releasing cytolytic compounds or by binding receptors on cancer cell membranes and inducing apoptosis of said cancer cells. Cytotoxicity is affected not only by signals that induce release of cytolytic compounds but also by signals that inhibit their release. An increase in cytotoxicity will therefore lead to more efficient killing of cancer cells, with less chance of the cancer cell dampening the cytotoxic activity of the NK, as mentioned above.

Cytokine-inducible SH2-containing protein (CIS) expression is induced by certain growth cytokines and is a key negative regulator of interleukin-15 (IL-15) signalling in natural killer (NK) cells. CIS is encoded by the CISH gene. CISH expression is associated with limited cell expansion and decreased cytotoxic activity against multiple cancer cell lines when maintained at low cytokine concentrations. As such, knocking out CISH expression in NK cells has recently been reported as beneficial for NK cell cytotoxicity against cancer cells (Zhu et al. 2020 “Metabolic Reprograming via Deletion of CISH in Human iPSC-Derived NK Cells Promotes In Vivo Persistence and Enhances Anti-tumor Activity” Cell: Vol. 27(2): pp. 224-237). That said, as with all genetic modifications, there are circumstances where the modification is more beneficial than others and it is imperative that there is sound scientific basis behind a therapy that utilises genetically modified cells.

As an alternative to cell-based therapies for cancer, other options are available such as small molecule therapies and antibody therapies.

There is nevertheless a need for new and improved cancer therapies that make use of different strategies (e.g., cell-based, antibody-based, small molecule etc.) to maximise the targeting and killing of cancer cells.

An object of the present invention is to provide NK cells and NK cell lines that target cancer cells with a more cytotoxic and/or persistent phenotype. A further object is to provide methods for producing modified NK cells and NK cell lines, compositions containing the cells or cell lines and uses of such in therapy, specifically the treatment of cancers. More particular embodiments aim to provide treatments for identified cancers. Specific embodiments aim at combining two or more therapies in a synergistic manner to enhance the cytotoxicity and persistence of the modified cells.

### SUMMARY OF THE INVENTION

There are provided herein modified NK cells and NK cell lines with a more cytotoxic phenotype, and methods of making the cells and cell lines. Also provided are compositions of modified NK cells and NK cell lines, and uses of said compositions for treating cancer. Cytotoxicity in this context, as above, refers to the killing of cancer cells.

The invention provides NK cells and NK cell lines in combination with immunomodulatory drugs for use in treating cancer, wherein the NK cells or NK cell lines have been modified to have reduced expression of CISH.

According to the invention, there are further provided methods of treating cancer, e.g. blood cancer, using immunomodulating drugs in combination with modified NK cells and cell lines, both generally and specifically, wherein the modified NK cells and lines are engineered to have reduced function of CIS. Optionally, in addition the cells lack expression of one or more checkpoint inhibitory receptors, express one or more TRAIL ligands, express one or more chimeric antigen receptors, express one or more growth-promoting cytokines and/or express one or more Fc receptors.

Diseases particularly treatable according to the invention include cancers, solid cancers and blood cancers, more particularly multiple myeloma. Tumours and cancers in humans in particular can be treated. References to tumours herein include references to neoplasms.

### DETAILS OF THE INVENTION

Accordingly, the present invention provides a natural killer (NK) cell or NK cell line in combination with an immunomodulatory drug for use in treating cancer, wherein the NK cell or NK cell line has been modified to have reduced expression of CISH (when compared to the same NK cell or NK cell line without the modification).

The present invention also provides a natural killer (NK) cell or NK cell line for use in treating cancer, wherein the NK cell or NK cell line has been modified to have reduced expression of CISH (when compared to the same NK cell or NK cell line without the modification), and wherein the NK cell or cell line is for administration in combination with an immunomodulatory drug.

The present invention further provides an immunomodulatory drug for use in treating cancer, wherein the immunomodulatory drug is for administration in combination with a natural killer (NK) cell or NK cell line which has been modified to have reduced expression of CISH (when compared to the same NK cell or NK cell line without the modification).

Together, the NK cells and NK cell lines of the invention will also be referred to as the NK cells (unless the context requires otherwise). Preferably, the NK cells are human NK cells.

As described in detail below in examples, NK cells and NK cell lines have been genetically modified so as to increase their cytotoxic activity, particularly against cancer. It is therefore preferable that the modification is to knock down or knock out expression of CISH.

Preferably, CISH expression is reduced by at least 50%, at least 75%, at least 90%, at least 95%, more preferably at least 99%, compared to the same NK cell or NK cell line without the modification (wildtype NK cell). Genetic knockout of CISH is preferably by CRISPR gene editing.

It is preferred that CISH expression is knocked out in the NK cell.

The preferred immunomodulatory drug of the invention is an Ikaros pathway modulator or a modulator of the cereblon E3 ligase complex. Preferably, the modulator is an immunomodulatory imide drug (IMiD) or a cereblon E3 Ligase Modulating Drug (CELMOD). In some contexts, the term CELMOD may be used in a manner that is intended to capture the IMiD class.

Preferably, the IMID is selected from Thalidomide, Lenalidomide, Iberdomide and Pomalidomide. More preferably, the IMID is selected from Lenalidomide and Iberdomide. Most preferably, the IMiD is Lenalidomide.

Preferably the CELMOD is selected from Iberdomide (CC-220) and CC-92480. Most preferably, the CELMOD is Iberdomide.

The cancer to be treated is preferably a blood cancer. Preferably the blood cancer is selected from multiple myeloma, acute myeloid leukaemia and acute lymphoblastic leukaemia. Optionally, the blood cancer is a B cell neoplasm.

Optionally, the cancer to be treated is preferably a solid cancer.

In a particularly preferred embodiment, the present invention provides an NK cell in combination with Lenalidomide for use in treating multiple myeloma, wherein the NK cell has been modified to have expression of the CISH gene knocked out.

A significant advantage of the invention lies in the unexpected synergy resulting from a combination cancer therapy comprising an immunomodulatory drug and an NK cell with reduced CISH expression. IMiDs and CELMoDs are known to degrade Ikaros and Aiolos (haemopoietic-specific zinc finger transcription factors that are important regulators of lymphocyte differentiation) by selective ubiquitination by the CRBN-CRL4 ubiquitin ligase (Kronke 2014 and Lu 2014). Work to date in this area has mainly been directed at investigating B and T cell development. However, it is known that Ikaros antagonises DNA binding by STAT5 in pre-B cells. STAT5 happens to target the CISH gene promoter and upregulate its expression, thus the inventors herein have elucidated that an IMiD/CELMOD cancer therapy inadvertently upregulates CISH expression in NK cells through the degradation of Ikaros. Interestingly, this also occurs in a STAT5 independent manner, due to a lack of direct transcriptional repression by Ikaros. IMiDs/CELMoDs preferred for use in the invention (of which Lenalidomide and Iberdomide are named examples) are hence those that result in upregulation of CISH expression, e.g., via degradation of Ikaros and/or Aiolos.

IMID-/CELMOD-induced upregulation of CISH expression in NK cells is an uncharacterised and unexpected side-effect, and the present inventors have devised a cancer therapy that takes advantage of this finding. By providing CISH-downregulated NK cells in combination with the IMID/CELMOD therapy, the NK cells can assert their maximal cytotoxic effect without being negatively impacted by upregulated CISH expression.

Hence, the immunomodulatory drug of the invention is an Ikaros-depleting agent.

A preferred feature of the invention is to provide modified NK cells and NK cell lines with an increased intrinsic capacity for rapid growth and proliferation in culture. This can be achieved, for example, by transfecting the cells to overexpress growth-inducing cytokines IL-2 and/or IL-15. Preferably, the cells are modified to express exogenous soluble IL-2 (sIL-2). Moreover, this optional alteration provides a cost-effective alternative to replenishing the growth medium with cytokines on a continuous basis. These cells can be used as intermediates to prepare significant quantities of cells for subsequent processing (e.g. to reduce their divisional capacity) prior to therapeutic application.

In other embodiments of the invention, NK cells are provided that also express or overexpress a TRAIL ligand. Preferably, the TRAIL ligand is a mutant (variant) TRAIL ligand. The resulting NK cells exhibit increased binding to TRAIL receptors and, as a result, increased cytotoxicity against cancers, especially solid cancers, in particular ovarian, breast and colorectal cancers, and blood cancers, in particular leukemias. The NK cells with this combined activity may also be effective in reducing cancer metastases.

The TRAIL mutants/variants preferably have lower affinity (or in effect no affinity) for ‘decoy’ receptors, compared with the binding of wild type TRAIL to decoy receptors. Such decoy receptors represent a class of TRAIL receptors that bind TRAIL ligand but do not have the capacity to initiate cell death and, in some cases, act to antagonize the death signaling pathway. Mutant/variant TRAIL ligands may be prepared according to WO 2009/077857.

The mutants/variants may separately have increased affinity for TRAIL receptors, e.g. DR4 and DR5. Wildtype TRAIL is typically known to have a KD of >2 nM for DR 4, >5 nM for DR 5 and >20 nM for the decoy receptor DcR1 (WO 2009/077857; measured by surface plasmon resonance), or around 50 to 100 nM for DR4, 1 to 10 nM for DR 5 and 175 to 225 nM for DcR 1 (Truneh, A. et al. 2000; measured by isothermal titration calorimetry and ELISA). Therefore, an increased affinity for DR4 is suitably defined as a KD of <2 nM or <50 nM, respectively, whereas an increased affinity for DR5 is suitably defined as a KD of <5 nM or <1 nM, respectively. A reduced affinity for decoy receptor DcR1 is suitably defined as a KD of >50 nM or >225 nM, respectively. In any case, an increase or decrease in affinity exhibited by the TRAIL variant/mutant is relative to a baseline affinity exhibited by wildtype TRAIL. The affinity is preferably increased at least 10%, at least 25%, at least 50%, at least 100%, more preferably at least 1000%, compared with that exhibited by wildtype TRAIL.

The TRAIL variant preferably has an increased affinity for DR5 as compared with its affinity for DR 4, DcR 1 and DcR 2. Preferably, the affinity is at least 1.5-fold, 2-fold, 5-fold, 10-fold, 100-fold, or even 1,000-fold or greater for DR 5 than for one or more of DR 4, DcR 1 and DcR 2. More preferably, the affinity is at least 1.5-fold, 2-fold, 5-fold, 10-fold, 100-fold, or even 1,000-fold or greater for DR 5 than for at least two, and The TRAIL variant preferably has an increased affinity for one or both of DR4 and DR5 as compared with its affinity for wildtype TRAIL. Preferably, the affinity is at least 1.5-fold, 2-fold, 5-fold, 10-fold, 100-fold, or even 1,000-fold or greater for DR 4 and/or DR5 than for wildtype TRAIL.

Further specific embodiments comprise a NK cell expressing a mutant TRAIL ligand that has reduced or no affinity for TRAIL decoy receptors. Further specific embodiments comprise a NK cell expressing a mutant TRAIL ligand that has reduced or no affinity for TRAIL decoy receptors and increased affinity for DR4 and/or DR5.

Binding affinity may be measured according to any suitable method known in the art. Preferably, binding affinity is measured using surface plasmon resonance, isothermal titration calorimetry or ELISA.

In certain embodiments, the TRAIL variant comprises at least one amino acid substitution at a position selected from the group consisting of 131, 149, 159, 160, 189, 191, 193, 195, 199, 200, 201, 203, 204, 212, 213, 214, 215, 218, 240, 251, 261, 264, 266, 267, 269, and 270.

In certain embodiments, the TRAIL variant comprises at least one substitution selected from the group consisting of G131R, G131K, R1491, R149M, R149N, R149K, S159R, G160E, Y189A, Y189Q, R191K, Q193H, Q193K, Q193S, Q193R, E195R, N199V, N199R, N199H, T200H, K201R, K201H, D203A, K204E, K204D, K204Y, K212R, Y213W, T214R, S215D, S215E, S215H, S215K, S215D, D218H, D218A, Y240A, K251D, K251E, K251Q, T261L, H264R, I266L, D267Q, D269A, D269H, and H270D.

In certain embodiments, the TRAIL variant comprises at least two substitutions selected from the group consisting of G131R, G131K, R149I, R149M, R149N, R149K, S159R, G160E, Y189A, Y189Q, R191K, Q193H, Q193K, Q193S, Q193R, E195R, N199V, N199R, N199H, T200H, K201R, K201H, D203A, K204E, K204D, K204Y, K212R, Y213W, T214R, S215D, S215E, S215H, S215K, S215D, D218H, D218A, Y240A, K251D, K251E, K251Q, T261L, H264R, I266L, D267Q, D269A, D269H, and H270D.

In certain embodiments, the TRAIL variant comprises at least three substitutions selected from the group consisting of G131R, G131K, R1491, R149M, R149N, R149K, S159R, G160E, Y189A, Y189Q, R191K, Q193H, Q193K, Q193S, Q193R, E195R, N199V, N199R, N199H, T200H, K201R, K201H, D203A, K204E, K204D, K204Y, K212R, Y213W, T214R, S215D, S215E, S215H, S215K, S215D, D218H, D218A, Y240A, K251D, K251E, K251Q, T261L, H264R, 266L, D267Q, D269A, D269H, and H270D.

In certain embodiments, amino acid substitution of the TRAIL variant is selected from the group consisting of G131R, G131K, R1491, R149M, R149N, R149K, S159R, G160E, Y189A, Y189Q, R191K, Q193H, Q193K, Q193S, Q193R, E195R, N199V, N199R, N199H, T200H, K201R, K201H, D203A, K204E, K204D, K204Y, K212R, Y213W, T214R, S215D, S215E, S215H, S215K, S215D, D218H, D218A, Y240A, K251D, K251E, K251Q, T261L, H264R, I266L, D267Q, D269A, D269H, H270D, T214R/E195R, T214R/D269H, Y189A/Q193S/N199V/K201R/Y213W/S215D, Y215D, Y213W/S215D, N199R/K201H, N199H/K201R, G131R/N199R/K201H, G131R/N199R/K201H/R1491 S159R/S215D, G131R/R149I/S159R/S215D, G131R/N199R/K201H/R1491/S159R/S215D, G131R/D218H, Y189Q/R191K/Q193R/H264R/1266L/D267Q, T261L/G160E, T261L/H270D, T261L/G160E/H270D, and T261L/G160E/H270D/T200H (use of “/” indicates multiple amino acid substitutions).

In certain embodiments, amino acid substitution of the TRAIL variant is selected based on the variant having an increased affinity for DR5; a substitution of this kind may be selected from the group consisting of D269H, E195R, T214R, D269H/E195R, T214R/E195R, T214R/D269H, N199V, Y189A/Q193S/N199V/K201R/Y213W/S215D, Y213W/S215D, D269A and Y240A.

In certain embodiments, amino acid substitution of the TRAIL variant is selected based on the variant having an increased affinity for DR4; a substitution of this kind may be selected from the group consisting of G131R, G131K, R149I, R149M, R149N, R149K, S159R, Q193H, W193K, N199R, N199R/K201H, N199H/K201R, G131R/N199R/K201H, G131R/N199R/K201H, G131R/N199R/K201H/R149I/S159R/S215D, G131R/R149I/S159R/S215D, G131R/D218H, K201R, K201H, K204E, K204D, K204L, K204Y, K212R, S215E, S215H, S215K, S215D, D218H, K251D, K251E, K251Q and Y189Q/R191K/Q193R/H264R/1266L/D267Q.

In certain embodiments, amino acid substitution of the TRAIL variant is selected based on the variant having a decreased affinity for TRAIL decoy receptors; a substitution of this kind may be selected from the group consisting of T261L, H270D, T200H, T261L/G160E, T261L/H270D, T261L/G160E/H270D, T261L/G160E /H270D/T200H, D203A and D218A.

Treatment of a cancer using modified NK cells expressing TRAIL or a TRAIL variant is optionally enhanced by administering to a patient an agent capable of upregulating expression of TRAIL death receptors on cancer cells. This agent may be administered prior to, in combination with or subsequently to administration of the modified NK cells. It is preferable, however, that the agent is administered prior to administering the modified NK cells. The agent upregulates expression of DR5 on cancer cells. The agent may optionally be a chemotherapeutic medication, e.g. a proteasome inhibitor, one of which is Bortezomib, and administered in a low dose capable of upregulating DR5 expression on the cancer. Other examples of DR5-inducing agents include Gefitinib, Piperlongumine, Doxorubicin, Alpha-tocopheryl succinate and HDAC inhibitors.

In certain embodiments of the invention, NK cells are provided that are further modified so as to have reduced or absent function of a checkpoint inhibitory receptor. NK cells may be produced that have one or more checkpoint inhibitory receptor genes knocked down/out. Preferably, these receptors are specific checkpoint inhibitory receptors. Preferably still, these checkpoint inhibitory receptors are one or more or all of TIGIT, CD96 (TACTILE), CD152 (CTLA4), CD223 (LAG-3), CD279 (PD-1), CD328 (SIGLEC7), SIGLEC9 and/or TIM-3. In other embodiments, NK cells are provided in which one or more inhibitory receptor signaling pathways are knocked out or exhibit reduced function—the result again being reduced or absent inhibitory receptor function. For example, signaling pathways mediated by SHP-1, SHP-2 and/or SHIP are knocked out by genetic modification of the cells.

It is preferred to reduce function of checkpoint inhibitory receptors over other inhibitory receptors, due to the expression of the former following NK cell activation. The normal or ‘classical’ inhibitory receptors, such as the majority of the KIR family, NKG2A and LIR-2, bind MHC class I and are therefore primarily involved in reducing the problem of self-targeting. Preferably, therefore, checkpoint inhibitory receptors are knocked out. Reduced or absent function of these receptors according to the invention prevents cancer cells from suppressing immune effector function (which might otherwise occur if the receptors were fully functional). Thus, a key advantage of these embodiments of the invention lies in NK cells that are less susceptible to suppression of their cytotoxic activities by cancer cells; as a result, they are useful in cancer treatment.

As used herein, references to inhibitory receptors generally refer to a receptor expressed on the plasma membrane of an immune effector cell, e.g. a NK cell, whereupon binding its complementary ligand resulting intracellular signals are responsible for reducing the cytotoxicity of said immune effector cell. These inhibitory receptors are expressed during both ‘resting’ and ‘activated’ states of the immune effector cell and are often associated with providing the immune system with a ‘self-tolerance’ mechanism that inhibits cytotoxic responses against cells and tissues of the body. An example is the inhibitory receptor family ‘KIR’ which are expressed on NK cells and recognize MHC class I expressed on healthy cells of the body.

Also as used herein, checkpoint inhibitory receptors are usually regarded as a subset of the inhibitory receptors above. Unlike other inhibitory receptors, however, checkpoint inhibitory receptors are expressed at higher levels during prolonged activation and cytotoxicity of an immune effector cell, e.g. a NK cell. This phenomenon is useful for dampening chronic cytotoxicity at, for example, sites of inflammation. Examples include the checkpoint inhibitory receptors PD-1, CTLA-4 and CD96, all of which are expressed on NK cells.

It is preferred in the invention not to reduce the function of inhibitory receptors that bind MHC class I.

The modified cells of the invention may also express a chimeric antigen receptor (CAR). The membrane-bound CAR typically comprises a targeting sequence (commonly an antibody-derived single-chain fragment (scFv)) and usually a hinge (to overcome steric hindrance issues), a spacer, a membrane-spanning element and a signalling endodomain.

The CAR preferably comprises a targeting region that binds an antigen selected from PD-L1, VEGF, VEGFR2, PD-1, MUC-1, CLL-1, TIM-3, CD19, EGFR, CD38, GD2, SLAMF7, CTLA4, CCR4, CD20, PDGFRa, HER2, CD33, CD30, CD22, CD79B, Nectin-4 and TROP2.

The CAR preferably comprises a targeting region that binds an antigen selected from HER2, TIM-3, MUC-1, CD38, CD96, CLL-1, SLAMF7 and CD19. Most preferably, the CAR binds an antigen selected from HER2, CD38 and MUC-1.

Preferably, the CAR targets CD38 and the NK cell expressing the CAR is additionally modified to have reduced expression of CD38. Preferably, expression of the CD38 gene is knocked out.

Examples of sequences that are known to bind aberrantly glycosylated MUC-1 can be used as the targeting sequence, such sequences including 5E5, SM3 and HMFG2, and are suitably incorporated in a CAR of the invention; preferably the CAR comprises the HMFG2 sequence. Nevertheless, further sequences for targeting aberrantly glycosylated MUC-1 may be identified through screening methods known in the prior art, wherein high affinity sequences can be used to produce CAR-NK cells targeting MUC-1.

Examples of sequences that are known to bind CD38 can be used as the targeting sequence, such sequences including daratumumab, isatuximab and those disclosed in WO 2018/104562, and are suitably incorporated in a CAR of the invention. It is preferred that the CD38 CAR has reduced affinity for CD38, compared to the affinity of daratumumab for CD38. Preferably, this reduced affinity is by at least 10%, at least 25%, more preferably at least 45%, compared to the affinity of daratumumab. Preferably, also, this reduced affinity is no more than 90%, no more than 75%, more preferably no more than 55%, compared to the affinity of daratumumab.

The CARs used in the NK cells of the invention may comprise or be linked to one or more NK cell costimulatory domains, e.g. CD28, CD134/OX 40, 4-1BB/CD137, CD3zeta/CD247, DAP12 or DAP10. Binding of the CAR to its antigen on a target cell thus promotes cytotoxic signals in the modified NK cell.

In an optional embodiment, the combination therapy of the invention may be used/administered further in combination with an antibody specific for an antigen on a cancer cell. It is preferred that the combination therapy of the invention is administered in combination with an antibody targeting an antigen selected from PD-L1, VEGF, VEGFR2, PD-1, MUC-1, CLL-1, TIM-3, CD19, EGFR, CD38, GD2, SLAMF7, CTLA4, CCR4, CD20, PDGFRa, HER2, CD33, CD30, CD22, CD79B, Nectin-4 and TROP2.

Preferably, the antibody used in combination with the therapy of the invention is selected from Atezolizumab, Avelumab, Bevacizumab, Brentuximab, Blinatumomab, Cemiplimab, Cetuximab, Daratumumab, Dinutuximab, Durvalumab, Elotuzumab, Enfortumab, Gemtuzumab, Ibritumomab, Ipilimumab, Inotuzumab, Isatuximab, Mogamulizumab, Necitumumab, Nivolumab, Obinutuzumab, Ofatumumab, Olaratumab, Panitumumab, Pembrolizumab, Pertuzumab, Polatuzumab, Ramucirumab, Rituximab, Sacituzumab, Tositumomab and Trastuzumab.

More preferably, the antibody used in combination with the therapy of the invention is Trastuzumab or Daratumumab

NK cells according to the invention may also be treated or pre-treated to render them incapable of division. This results in further reduced lifetime in circulation in the patient, e.g. in comparison with T cells, further mitigating the risks above, and also with reduced or absent propensity to form tumours in a patient.

NK cells of the invention are for use in therapy, especially in treating cancer in a patient. The cancer is suitably a cancer expressing CD38. Preferably, the cancer is a myeloma, leukaemia or solid cancer. It is particularly preferred that the cancer is multiple myeloma.

NK cell resistant cancers in general are well-known in the art (Pardoll, D. M. Immunity (2015) 42:605-606). Sensitivity of cancer cells to NK cell-mediated killing is determined by a number of factors. There exists a balance of positive and negative signals, largely delivered through membrane receptors on NK cells interacting with ligands on cancer cells. It is often the balance of expression of the ligands for these receptors that determines whether a cancer is sensitive or resistant to killing by NK cells (Yokoyama, W. M. Immunol Res (2005) 32:317-325).

Cancer sensitivity to NK cell-mediated cytotoxicity is generally understood as falling into one of the following categories: highly resistant, resistant, sensitive and highly sensitive. In the laboratory, cancer cells can be screened for their sensitivity to NK cell-mediated cytotoxicity through the use of cytotoxicity assays. Each category is then understood as corresponding to the percentage of cancer cells that are killed during exposure to NK cells at a specific effector: target (E: T) ratio and for a specific amount of time.

In examples of the invention, a cancer is said to be highly resistant to NK cell-mediated killing if ≤25% of the cancer cells are killed after incubation with NK cells for up to 15 hours at an E: T ratio of up to 5:1. Preferably, the NK cells are KHYG-1 cells.

In examples of the invention, a cancer is said to be resistant to NK cell-mediated killing if ≤50% of the cancer cells are killed after incubation with NK cells for up to 15 hours at an E: T ratio of up to 5:1. Preferably, the NK cells are KHYG-1 cells.

In examples of the invention, a cancer is said to be sensitive to NK cell-mediated killing if >50% of the cancer cells are killed after incubation with NK cells for up to 15 hours at an E: T ratio of up to 5:1. Preferably, the NK cells are KHYG-1 cells.

In examples of the invention, a cancer is said to be highly sensitive to NK cell-mediated killing if ≥75% of the cancer cells are killed after incubation with NK cells for up to 15 hours at an E: T ratio of up to 5:1. Preferably, the NK cells are KHYG-1 cells.

In preparing genetically modified NK cells, the modification may occur before the cell has differentiated into an NK cell. For example, pluripotent stem cells (e.g. iPSCs) can be genetically modified then differentiated to produce genetically modified NK cells with increased cytotoxicity.

Optional features of the invention include providing further modifications to the NK cells and NK cell lines described above, wherein, for example, a Fc receptor (which can be CD16, CD32 or CD64, including subtypes and derivatives) is expressed on the surface of the cell. In use, these cells can show increased recognition of antibody-coated cancer cells and improved activation of the cytotoxic response.

Further optional features of the invention include adapting the modified NK cells and NK cell lines to better home to specific target regions of the body. NK cells of the invention may be targeted to specific cancer cell locations. In preferred embodiments for treatment of blood cancers, NK effectors of the invention are adapted to home to bone marrow. Specific NK cells are modified by fucosylation and/or sialylation to home to bone marrow. This may be achieved by genetically modifying the NK cells to express the appropriate fucosyltransferase and/or sialyltransferase, respectively. Increased homing of NK effector cells to tumour sites may also be made possible by disruption of the tumour vasculature, e.g. by metronomic chemotherapy, or by using drugs targeting angiogenesis (Melero et al, 2014) to normalize NK cell infiltration via cancer blood vessels.

The invention further provides a method of treating cancer in a patient, comprising:
a) administering an immunomodulatory drug, e.g. an IMiD, to a patient in a therapeutically effective dosage; and
b) administering an NK cell to a patient in a therapeutically effective dosage,
wherein the NK cell has been modified to have reduced expression of CISH.

In related methods of the invention, there is provided a method of treating cancer in a patient, comprising:
administering an immunomodulatory drug, e.g. an IMiD, to a patient in a therapeutically effective dosage;
wherein the patient has already been administered or is being administered or will be administered an NK cell in a therapeutically effective dosage, wherein the NK cell has been modified to have reduced expression of CISH.
In other related methods of the invention, there is provided a method of treating cancer in a patient, comprising:
administering an NK cell to a patient in a therapeutically effective dosage, wherein the NK cell has been modified to have reduced expression of CISH;
wherein the patient has already been administered or is being administered or will be administered an immunomodulatory drug, e.g. an IMiD in a therapeutically effective Preferably, the CISH gene has been knocked out in the NK cell.
Administration of the IMID/CELMOD may be simultaneously with, prior to or subsequent to administration of the NK cell. In preferred therapies the administration is substantially simultaneous.
Further provided are compositions (e.g. pharmaceutical compositions) comprising NK cells and an immunomodulatory drug, e.g. an IMiD, wherein the NK cells have been modified to have reduced expression of CISH.

Preferably, the CISH gene has been knocked out in the NK cells.

Modified NK cells, NK cell lines and compositions thereof described herein, above and below, are suitable for treatment of cancer, in particular cancer in humans, e.g. for treatment of cancers of blood cells or solid cancers. The NK cells and derivatives thereof are preferably human NK cells. For human therapy, human NK cells are preferably used. The invention also provides methods of treating cancer in humans comprising administering an effective amount of the NK cells or compositions comprising the same.

Various routes of administration will be known to the skilled person to deliver active agents and combinations thereof to a patient in need. Administration of the modified NK cells can be systemic or localized, such as for example via the intraperitoneal route. In other embodiments, active agent is administered more directly. Thus, administration can be directly intratumoural, suitable especially for solid tumours.

NK cells in general are believed suitable for the methods, uses and compositions of the invention. As per cells used in certain examples herein, the NK cell can be a NK cell obtained from a cancer cell line. Advantageously, a NK cell, preferably treated to reduce its tumorigenicity, for example by rendering it mortal and/or incapable of dividing, can be obtained from a blood cancer cell line and used in methods of the invention to treat blood cancer.

To render a cancer-derived NK cell more acceptable for therapeutic use, it is generally treated or pre-treated in some way to reduce or remove its propensity to form tumours in the patient. Specific modified NK cell lines used in examples are safe because they have been rendered incapable of division; they are irradiated and retain their killing ability but die within about 3-4 days. Specific cells and cell lines are hence incapable of proliferation, e.g. as a result of irradiation. Treatments of potential NK cells for use in the methods herein include irradiation to prevent them from dividing and forming a tumour in vivo and genetic modification to reduce tumorigenicity, e.g. to insert a sequence encoding a suicide gene that can be activated to prevent the cells from dividing and forming a tumour in vivo. Suicide genes can be turned on by exogenous, e.g. circulating, agents that then cause cell death in those cells expressing the gene. A further alternative is the use of monoclonal antibodies targeting specific NK cells of the therapy. CD52, for example, is expressed on KHYG-1 cells and binding of monoclonal antibodies to this marker can result in antibody-dependent cell-mediated cytotoxicity (ADCC) and KHYG-1 cell death.

As discussed in an article published by Suck et al, 2006, cancer-derived NK cells and cell lines are easily irradiated using irradiators such as the Gammacell 3000 Elan. A source of Cesium-137 is used to control the dosing of radiation and a dose-response curve between, for example, 1 Gy and 50 Gy can be used to determine the optimal dose for eliminating the proliferative capacity of the cells, whilst maintaining the benefits of increased cytotoxicity. This is achieved by assaying the cells for cytotoxicity after each dose of radiation has been administered.

There are significant benefits of using an irradiated NK cell line for adoptive cellular immunotherapy over the well-established autologous or MHC-matched T cell approach. Firstly, the use of a NK cell line with a highly proliferative nature means expansion of modified NK cell lines can be achieved more easily and on a commercial level. Irradiation of the modified NK cell line can then be carried out prior to administration of the cells to the patient. These irradiated cells, which retain their useful cytotoxicity, have a limited life span and, unlike modified T cells, will not circulate for long periods of time causing persistent side-effects.

Additionally, the use of allogeneic modified NK cells and NK cell lines means that MHC class I expressing cells in the patient are unable to inhibit NK cytotoxic responses in the same way as they can to autologous NK cytotoxic responses. The use of allogeneic NK cells and cell lines for cancer cell killing benefits from the previously mentioned GVL effect and, unlike for T cells, allogeneic NK cells and cell lines do not stimulate the onset of GVHD, making them a preferred option for the treatment of cancer via adoptive cellular immunotherapy.

### EXAMPLES

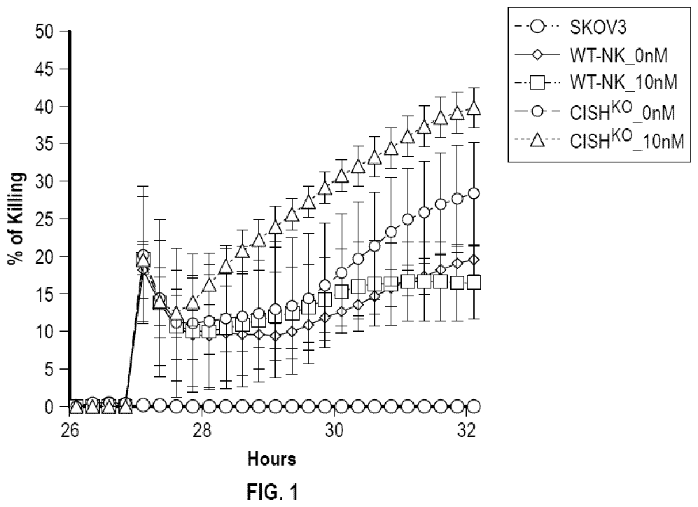

The present invention is now described in more and specific details in relation to the production of NK cells that are modified to exhibit more cytotoxic activity against a range of cancer cells and their uses in the combination therapy of the invention, with particular reference to the accompanying drawings, wherein:
FIG. 1 shows that CISH KO NK cells work in synergy with IMiD, iberdomine, resulting in enhanced cytotoxic properties against cancer cells.

For related examples of knockout/knockdown of inhibitory receptor function and knock in of TRAIL/mutant TRAIL we refer to WO 2017/017184, the contents of which are incorporated herein by reference.

For related examples of CD38 CAR expression we refer to WO 2018/104562, the contents of which are incorporated herein by reference.

For related examples of MUC-1 CAR expression we refer to WO 2019/101998, the contents of which are incorporated herein by reference.

### Example 1

### Design Protocol for Knockout of CISH in NK Cells

NK cells are prepared as follows, having CIS function removed. gRNA constructs are designed and prepared to target the CISH gene in NK cells. CRISPR/Cas9 genome editing is then used to knock out CISH expression.
A total of 3 gRNA candidates are selected for the CISH gene and their cleavage efficacies in primary expanded NK cells determined. The cells are electroporated with the gRNA: Cas9 ribonucleoprotein (RNP) complex using Maxcyte® GT and subsequently knockout of CISH is analysed by flowcytometry. The cleavage activity of the gRNA is also determined using an in vitro mismatch detection assay. T7E1 endonuclease recognizes and cleaves non-perfectly matched DNA, allowing the parental CISH gene to be compared to the mutated gene following CRISPR/Cas9 transfection and non-homologous end joining (NHEJ).

The gRNA with highest KO efficiency is selected for further experiments to knockout CISH in primary NK cells, NK cell lines or CD34+ progenitors (e.g. iPSCs for subsequent differentiation into and expansion of NK cells). Knockout of CISH is determined by flow cytometry based assays.

### Example 2

### Knockout of Cish in Nk Cells

### Materials

1. Enriched cord blood-derived NK cells.
2. NK-MACS medium, Premium grade IL-15, anti-CD3 VioBlue, Anti-CD56VB515, Inside Stain Kit, MACSquant 16 (Miltenyi Biosciences).
3. Human Serum Albumin (Sigma).
4. Gene knockout Kit V2 for CISH and Cas9 recombinant protein (Synthego).
5. Cloudz ™ Cell Activation Kits (R&D systems).
6. Anti-CISH antibody (D4D9) (Cell Signaling Technology).
7. Electroporation buffer (EB buffer), OC-100X2 processing assembly (PA), MaxCyte ATx electroporation system (Maxcyte).
8. Solu-Cortef (Hydrocortisone) (Pfizer).
9. Ficoll (Cytiva).

### Protocol

Cord Blood Mononuclear Cells (CBMCs) were isolated by the Ficoll method and enriched for 15 days in NK-MACS medium with 10% human serum albumin, CD2/NKp46 microsphere (Cloudz™), anti-CD3, anti-CD16, IL-15 and hydrocortisone.

NK cell enrichment of over 90% was confirmed by FACS (MACSquant 16) analysis by staining with anti-CD3 and anti-CD56 antibodies. CBNK cells were washed twice with Maxcyte electroporation buffer (EB buffer) 300 xg for 10mins and 5 million cells were taken in 75 μl of EB buffer.

sgRNA for CISH was dissolved in nuclease-free TE buffer to get a stock of 200 μM. 1200 pmol of sgRNA was mixed with 160 pmol Cas9 at a 7.5:1 ratio. The volume was adjusted with EB buffer to 25 μl and incubated at room temperature for 10 mins for formation of RNP complex. 25 μl RNP complex was mixed with 75 μl CBNK cells and transferred to the OC-100X2 processing assembly. The cells were electroporated using the NK5 protocol and left 15 mins to rest at room temperature. No RNP was used as a control.

Electroporated cells were transferred to the Grex 6 well plate in NK-MACS medium with 10% AB and 20ng/ml IL-15 and incubated at 37° C. in a 5% CO₂ incubator for 13 days. The cells were then analysed for CISH knockout every 3-4 days. Intracellular expression of CISH was analysed using Inside Stain Kit and an anti-CISH antibody.

Successful knockout of CISH expression in the NK cells was observed.

The NK cells exhibited superior proliferation and cytokine production compared to unmodified control NK cells, resulting in an overall improved cytotoxic phenotype.

### Example 3

### CISH KO NK Cells in Combination With Imids for Myeloma Therapy

The following protocol was developed for use in treating patients with multiple myeloma. Nevertheless, it is apparent that the invention is suitable for treating patients with many different cancers.

Following diagnosis of a patient with multiple myeloma, an aliquot of CISH KO NK cells is thawed and cultured prior to administration to the patient in an effective dose. The aliquoted cells may be additionally modified as described elsewhere herein. Alternatively, a transient transfection can be prepared using e.g. viral means, electroporation etc. For electroporation, the MaxCyte Flow Electroporation platform offers a suitable solution for achieving fast large-scale transfections in the clinic. After NK cells are effectively modified, they can be administered intravenously to the patient.

Prior to, simultaneously with or subsequent to administration of the CISH KO NK cells, Lenalidomide is administered in an effective dose to the patient.

### Example 4

Enhanced Cytotoxicity of CISH KO NK Cells in Combination With Ikaros Pathway Modulators Cord blood-derived NK cells were isolated for use in wildtype form or for use in the presence of a CISH gene knockout (as per Example 2).

The cytotoxicity of wildtype (WT) or CISH KO NK cells alone, or with the addition of iberdomide (10 nM), against the SKOV-3 GFP ovarian cancer target cells was evaluated in the following experimental setup. Human ovarian cancer target cells were plated in a 96 well eSight plate and allowed to adhere before the addition of fresh NK cells at 1:1 effector: target (E: T) ratio for 10 hours. The percentage of killing was calculated using the RTCA eSight system based on cell impedance. The experiments were conducted using 1) WT control NK cells, 2) WT NK cells +10 nM iberdomide, 3) NK cells with CISH KO only, 4) NK cells with CISH KO +10 nM iberdomide.

As can be seen from FIG. 1, iberdomide treated WT NK cells did not improve the percentage of killing in comparison to WT NK cells alone, with both groups achieving between 15-20% killing at 32 hours. CISH KO NK cells alone achieved approximately 27% killing at 32 hours, however the composition of CISH KO NK cells with iberdomide significantly enhanced the percentage of killing to approximately 40%.

These data demonstrate that a genetic knockout of CISH in NK cells works synergistically with iberdomide, resulting in a significantly enhanced cytotoxic NK cell phenotype.

The invention thus provides NK cells in combination with immunomodulatory drugs, as well as uses of this combination in cancer therapy.

1-14. (canceled)

15. A method of treating cancer in a patient, the method comprising administering to the patient a natural killer (NK) cell or NK cell line in combination with a cereblon E3 Ligase Modulating Drug (CELMOD) or an immunomodulatory imide drug (IMiD), wherein the NK cell or NK cell line has been modified to have reduced expression of CISH compared to the same NK cell or NK cell line without the modification

16. The method of claim 15, wherein the IMiD is selected from Lenalidomide and Iberdomide.

17. The method of claim 15, wherein the IMiD is Iberdomide.

18. The method of claim 15, wherein the modification is to genetically knock down expression of CISH.

19. The method of claim 15, wherein the modification is to genetically knock out expression of CISH.

20. The method of claim 15, wherein CISH expression is reduced by at least 50% compared to the same NK cell or NK cell line without the modification.

21. The method of claim 15, wherein CISH expression is reduced by at least 90% compared to the same NK cell or NK cell line without the modification.

22. The method of claim 15, wherein the NK cell or NK cell line is further modified to express IL-15, preferably in soluble form (sIL-15).

23. The method of claim 15, wherein the NK cell or NK cell line is further modified to express a TRAIL variant with increased affinity for TRAIL death receptors.

24. The method of claim 15, wherein the NK cell or NK cell line is further modified to express a chimeric antigen receptor (CAR) that binds HER2, TIM-3, CD19, CD38, MUC-1, CLL-1, SLAMF7 and/or CD96.

25. The method of claim 15, wherein the cancer is a blood cancer.

26. The method of claim 25, wherein the blood cancer is selected from multiple myeloma, acute myeloid leukaemia and acute lymphoblastic leukaemia.

27. The method of claim 15, wherein the cancer is a solid cancer.

28. The method of claim 15, wherein the CELMOD or IMiD is Ikaros-depleting.

29. A pharmaceutical composition comprising an IMiD or CELMOD and an NK cell, wherein CISH expression has been knocked out in the NK cell.

30. The pharmaceutical composition of claim 29, wherein the IMiD or CELMOD is Ikaros-depleting.

31. The pharmaceutical composition of claim 29, wherein the IMiD is selected from Lenalidomide and Iberdomide.

32. The pharmaceutical composition of claim 29, wherein the IMiD is Iberdomide.

33. The pharmaceutical composition of claim 29, wherein the NK cell is further modified to express IL-15, preferably in soluble form (sIL-15).

34. The pharmaceutical composition of claim 29, wherein the NK cell is further modified to express a chimeric antigen receptor (CAR) that binds HER2, TIM-3, CD19, CD38, MUC-1, CLL-1, SLAMF7 and/or CD96.
